(19) United States
(12) Patent Application Publication
(10) Pub. No.: US 2006/0095073 A1
(43) Pub. Date: May 4, 2006

(54) Arterial compression device and method

(76) Inventors: Robert Beto, Morgantown, WV (US); Chi Lam Yau, Dover, NH (US); Mark MacMahon, Portland, ME (US)

Correspondence Address: Kevin M. Farrell;Pierce Atwood, Suite 350, One New Hampshire Avenue, Portsmouth, NH, 03801, US

(21) Appl. No.: 10/981,395

(22) Filed: Nov. 4, 2004

(51) Int. Cl. A61B 17/00

(52) U.S. Cl. 606/201

(57) ABSTRACT

The present invention includes a device for inducing hemostasis in an artery, such as the femoral artery, and a method of doing the same. The device includes a base, vertical and horizontal members, an angular selector, and a compression arm adapted for providing constant mechanical pressure to a wound site. The angular selector and compression arm coordinate to maximize the device's degrees of freedom such that the pressure applied is perpendicular to the wound site for maximum efficacy. The compression arm includes a compressor and a cam that are used in concert for providing an optimal pressure that is easily referenced and can be reproduced by the attending medical personnel.

### BACKGROUND OF THE INVENTION

1. Field of the Invention

The present invention relates generally to the field of cardiovascular intervention, and more particularly to the field of devices and materials for inducing hemostasis in arteries during invasive procedures.

2. Summary of the Related Art

It is common in cardiac procedures to visualize arteries and veins with contrast medium. During heart catheterization and angiography for example, a surgeon will typically enter the cardiac system via the femoral artery in the groin. However, investigations via the femoral artery are especially difficult because of the high pressure against the artery wall. In a typical procedure, a surgeon will make a small incision in the femoral artery using a small diameter instrument. A guide is inserted into the incision and into the artery, and a catheter is threaded over the guide into the artery. Thereafter, the guide is removed and contrast medium is injected through the catheter into the artery.

After the investigation is completed, the catheter is withdrawn, leaving a wound in the femoral artery. Due to the high pressure within the femoral artery, the bleeding from the wound can be significant. A number of methods and devices have been developed to curtail this bleeding. For example, physicians may stem the bleeding manually by pressing at least one finger against a compressive bandage laid on the wound for about 20 minutes. This method is not satisfactory for a number of reasons. First, it is inconvenient for both patients and physicians and also requires valuable physician time. Furthermore, it is difficult for the physician to maintain a constant pressure for such a long period of time. Lastly, as these procedures are repeated many times a day, the repetitive strain on the hands of the physician will inevitably take its toll, potentially affecting the quantity of care made available to patients.

Several mechanical devices have also been proposed to curtail the bleeding from catheterization in the femoral artery. In general, the principle of these devices is the same: an external pressure is applied near the incision site in the femoral artery for about 20 minutes following completed catheterization. The pressure is to be set high enough to stop bleeding, but not so high that the blood flow is cut off down to the leg and foot.

However, although the devices themselves are designed to provide pressure against an incision site, the design and adaptability of these devices is limited. It can be appreciated that the cross-section of any portion of the human body, but most particularly the femoral region, is oval in shape. Accordingly, as the human body is not planar, there are 360 degrees of normal lines around any body surface. In spite of this fact, the existing art typically applies any pressure against the femoral incision in a downward direction. This direction is not necessarily perpendicular to the femoral artery, and thus any pressure placed on the artery will inevitably result in tangential and shearing forces that could cause discomfort to the patient and result in inadequate hemostasis and/or vascular complications.

Moreover, as the existing art is limited in its angular approach to applying pressure to the artery, the idiosyncrasies of individual patients can limit its effectiveness. These types of procedures are performed on individuals having 7% body fat as well as 37% body fat, and the accessibility of the femoral artery can vary greatly depending on each patient's body composition, shape, and muscle tone. The existing art does not include a method of ergonomically engaging the femoral artery in a manner that is adaptable for differing body shapes and sizes while maximizing patient comfort. There is a need in the art for such a device.

### SUMMARY OF THE INVENTION

Accordingly, the present invention includes a device for inducing hemostasis in an artery. The device includes a base, a vertical member coupled to the base, a horizontal member slidably coupled to the vertical member, an angular selector slidably coupled to the horizontal member, a compression arm rotatably coupled to the angular selector, and a pressure pad coupled to the compression arm wherein the compression arm is selectively positioned along a horizontal axis, a vertical axis, and an angular position such that the pressure pad applies pressure to an artery. The device of the present invention may be used as a femoral artery clamp for post-catheterization of a wound site during cardiac procedures.

The horizontal member and angular selector include a number of locking mechanisms that are used to secure the device in position. The compression arm includes a compressor and a cam that are used in concert for providing an optimal force to drive the pressure pad. The compressor may include a spring mechanism for supplying a first amount of force, while the cam is adjustable for fine-tuning the amount of force from the compressor.

The pressure pad defines a number of surfaces that approximate the profile of a surgeon's hands, including both a convex portion and a planar portion for applying pressure in more than one place along the artery. The pressure pad is optionally constructed of a transparent material such that the physician can more easily see the contact site. The present invention also includes an area upon which a coagulant is placed accelerating hemostasis. The present invention also includes a method of inducing hemostasis in an artery using a mechanism.

The foregoing is intended as a summary of the novel and useful features of the present invention. Further aspects, features and advantages of the invention will become apparent from consideration of the following detailed description and the appended claims when taken in connection with the accompanying drawings.

### BRIEF DESCRIPTION OF THE DRAWINGS

FIG. 1 is a perspective view of a femoral artery clamp in accordance with the present invention.

FIG. 2 is a perspective view of a locking horizontal member in accordance with the present invention.

FIG. 3 is an exploded view of a locking horizontal member in accordance with the present invention.

FIG. 4 is a perspective view of a compression arm in accordance with the present invention.

FIG. 5 is an exploded view of a compression arm in accordance with the present invention.

FIG. 6 is a perspective view of the femoral artery clamp of the present invention showing a typical patient in phantom.

FIG. 7 is a cross-sectional view of the femoral artery clamp of the present invention showing a cross-sectional portion of a patient's body in phantom.

### DETAILED DESCRIPTION OF THE PRESENT INVENTION

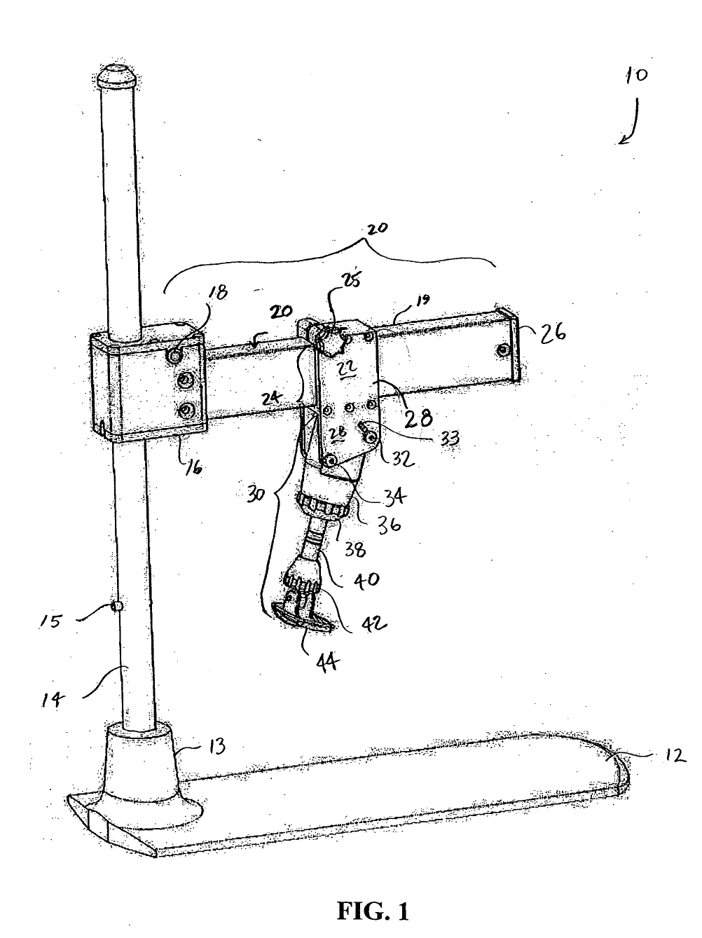

The present invention is best described with reference to the drawings. FIG. 1 is a perspective view of a femoral artery clamp 10 in accordance with the present invention. It should be understood that the present invention is not limited in its utility to curtailing bleeding in the femoral artery alone. Rather, the present invention is readily adaptable for a range of surgical needs that require a means for inducing hemostasis. Accordingly, the femoral artery clamp 10 will also be referred to as a device for inducing hemostasis in an artery.

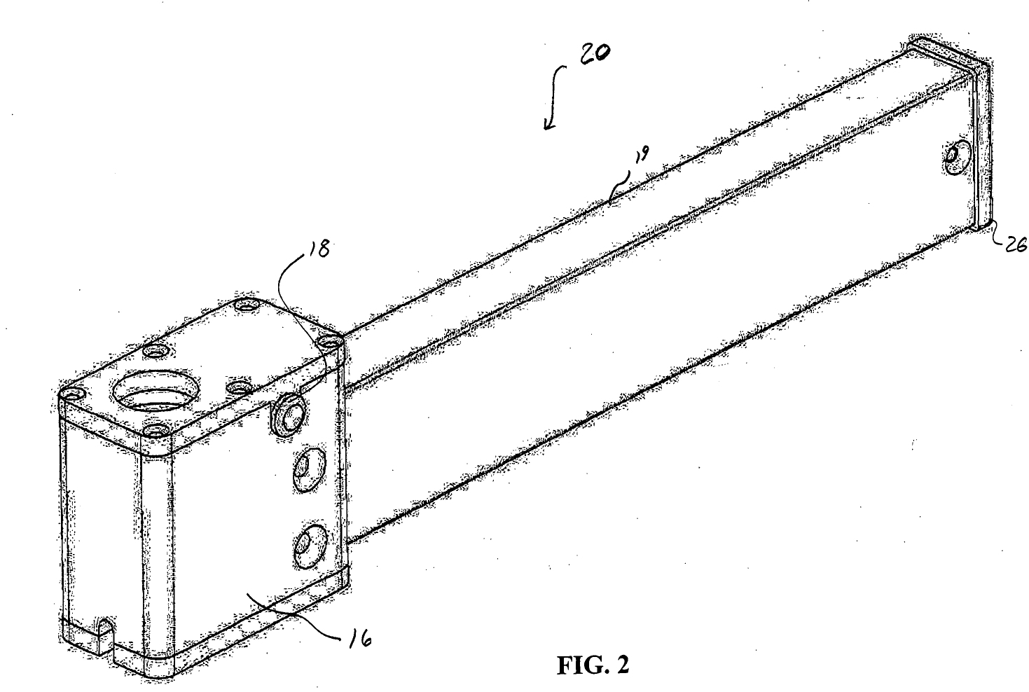

The device 10 is comprised generally of a base 12 defining a substantially planar surface. An anchor 13 is adapted for connecting a vertical member 14 to the base 12. A horizontal member 20 including a coupler 16 and a shaft 19 is coupled to the vertical member 14 by the coupler 16. On an opposite end, the horizontal member 20 is buttressed by a cap 26. FIG. 2 is a perspective view of the horizontal member 20 showing the coupler 16, the shaft 19 and the cap 26 in greater detail.

Returning to FIG. 1, the coupler 16, and thus the horizontal member 20, is slidably coupled to the vertical member 14 with a first locking mechanism 18 adapted for selectively locking the horizontal member 20 in place. A pin 15 is disposed on the vertical member 14 for preventing the horizontal member 20 from sliding too low on the vertical member 14 and further preventing the horizontal member 20 from rotating about the vertical member 14 when stowed.

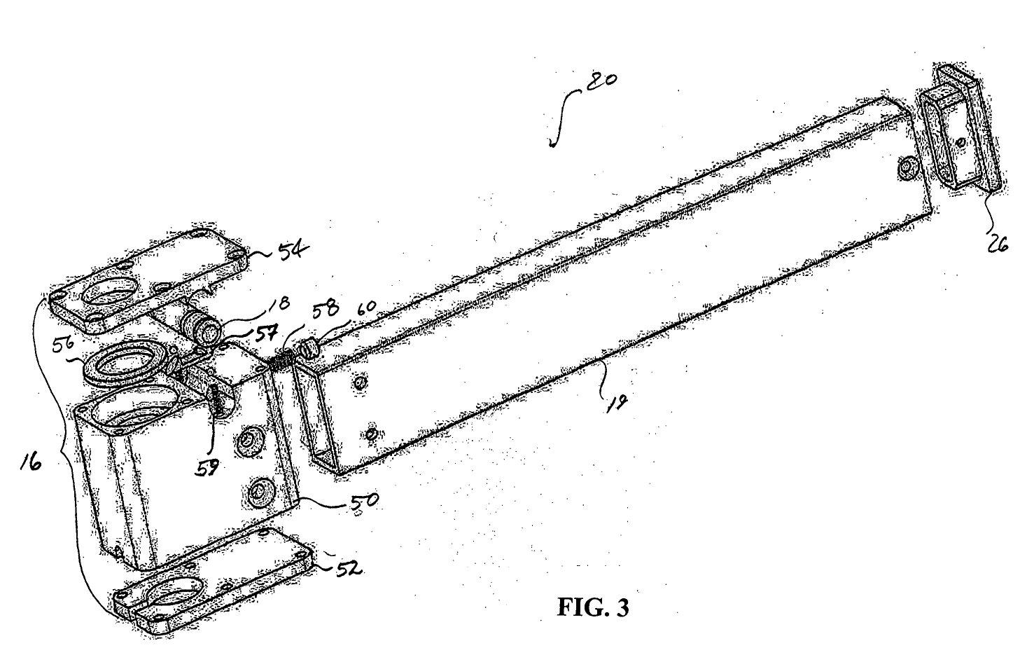

FIG. 3 is an exploded view of the horizontal member 20 in accordance with the present invention. The coupler 16 includes a bottom portion 52, a top portion 54 and a deformable washer 56. The deformable washer 56 is a rigid structure that provides a predetermined amount of resistance for holding the horizontal member 20 in place. The first locking mechanism 18 interacts with a series of springs 58, 59 for selectively moving a deforming pin 57. The spring 58 is contained within the coupler 16 by a plug 60. When the first locking mechanism 18 is in an open position, the springs 58, 59 are compressed and the deforming pin 57 is disengaged from the deformable washer 56, thus permitting the vertical movement of the coupler 16 about the vertical member 14. In a closed position, the first locking mechanism 18 allows the springs 58, 59 to press the deforming pin 57 into the deformable washer 56, restricting the movement of the coupler 16 about the vertical member 14.

It is noteworthy that even in the closed position, the first locking mechanism 18 does not prevent some movement of the horizontal member 20. On the contrary, should a patient move or be inadvertently shaken during a hemostatic procedure, the present invention is adapted to be reflexive and permit some movement for the patient's safety and comfort.

Returning now to FIG. 1 and the perspective view of the device 10 of the present invention, an angular selector 24 is slidably coupled to the horizontal member 20 and secured thereto by a second locking mechanism 25. The angular selector 24 includes a first portion 22 that is slidable with respect to the horizontal member 20, and a second portion 28 that is adapted to receive a compression arm 30. The compression arm 30 is rotatably coupled to the angular selector 24 at the second portion by an axle 34 and a third locking mechanism 32 that is disposable within a plurality of ports 33 across a range of angles. In one embodiment, the plurality of ports 33 is a series of holes defining an arc on the surface of the second portion 28. In alternate embodiments, the plurality of ports 33 may be a curvilinear slot through which the third locking mechanism 32 may slide until locked.

In any embodiment, the present invention functions by locking the third locking mechanism 32 in a selected port 33 such that the compression arm 30 will define an angle with respect to the horizontal member 20. The compression arm 30 may be locked in any position within a range of zero to 80 degrees with respect to the vertical member 14. Most preferably, the third locking mechanism is adapted to provide the compression arm 30 with a range from zero to sixty degrees with respect to the vertical member 14.

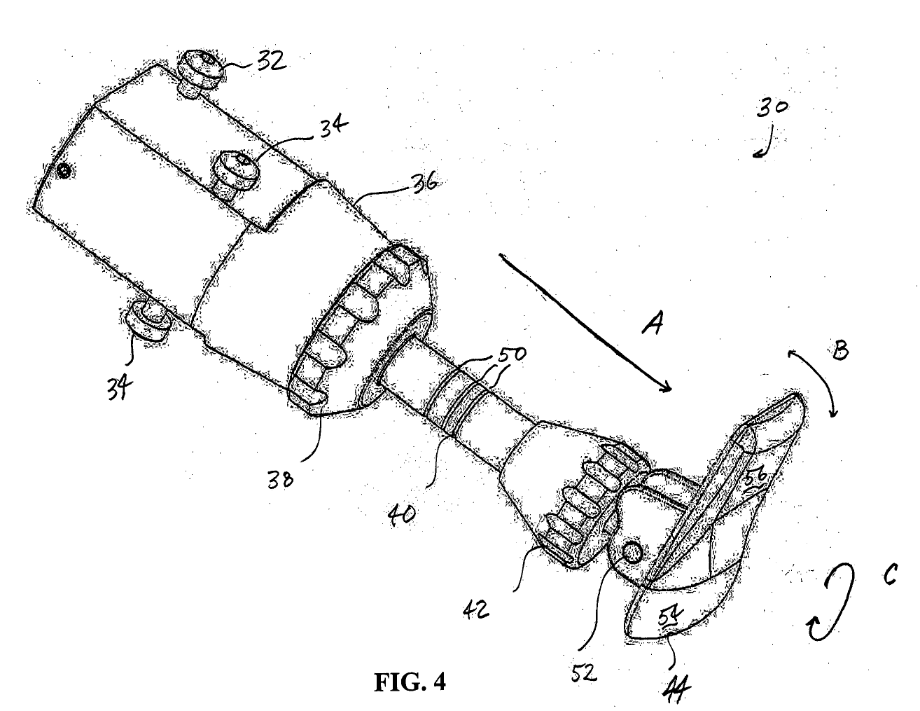

Referring now to FIGS. 1 and 4, the compression arm 30 generally defines a compressor 36 and a fifth locking mechanism, such as a cam 42, which are coupled by a rod 40. Preferably, the rod 40 has a series of markers 50, such as colored bands, which indicate that a predetermined spring force is being applied. The compressor 36 provides a substantial amount of the force exerted by the compression arm 30, adjusted by the fourth locking mechanism 38. The cam 42 provides a lesser amount of the force exerted by the compression arm 30, adjusted by the fifth locking mechanism 42. Both the compressor 36 and the cam 42 produce a force in the direction of a longitudinal axis mirrored by arrow A.

A pressure pad 44 is disposed at the end of the compression arm 30 for applying direct pressure to the wound site. The pressure pad 44 is coupled to the compression arm 30 such that it is rotatable along arrow C and about arrow A. The pressure pad 44 is also pivotable with respect to the compression arm 30 along arrow B. Thus, the pressure pad 44 is movable to some degree along arrows A, B, and C, and thus has a virtually limitless range of motion, making it readily adaptable to a number of surgical procedures.

The pressure pad 44 defines a first region 54 that is substantially convex in shape, and a second region 56 that is substantially planar in shape. In a preferred embodiment, the first region 54 accurately approximates the size of the fingers used by a physician to apply pressure to the arterial wall. It is noteworthy that both the first region 54 and the second region 56 are adapted to engage the arterial wall, thus providing multiple points of contact and dispersing the force supplied by the compression arm 30 over a greater surface area. In a preferred embodiment, the pressure pad 44 is made of a transparent material for allowing the physician to precisely place the device 10 over the wound site. In another preferred embodiment, the pressure pad 44 is coated with a coagulating material (not shown) that will accelerate the hemostatic process on contact with the wound site.

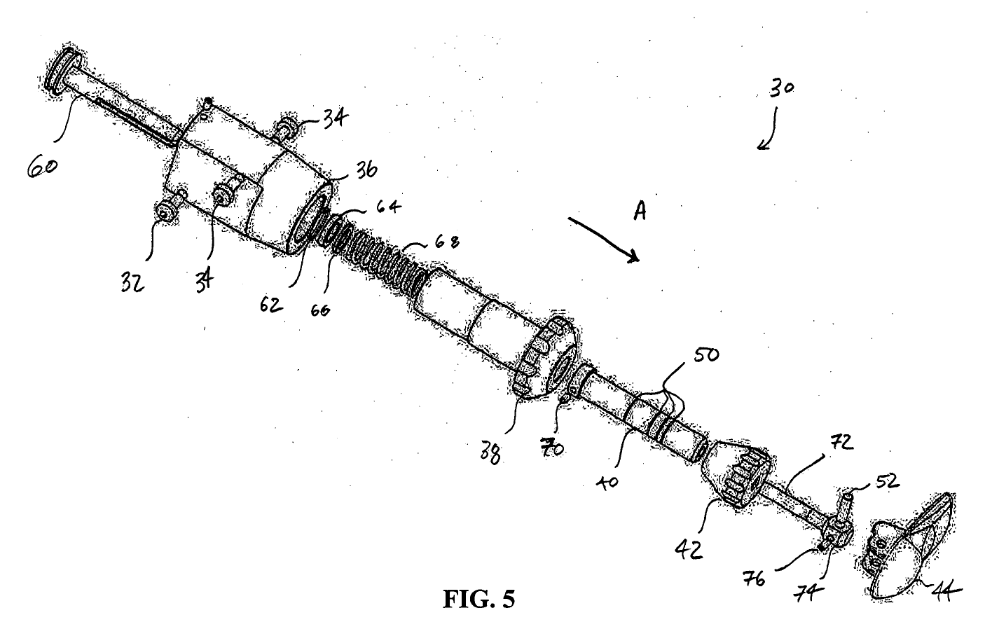

Further details of the compression arm 30 are visible in the exploded view of FIG. 5. The compressor 36 defines a cavity for receiving a spring 68 on one end and a plug 60 to buttress the spring 68 on another end. A series of bushings 62, 64, 66 are provided at the junction of the spring 68 and the plug 60; and therefore the spring 68 is contained within the fourth locking mechanism 38 buttressed by the bushings 62, 64, 66 and the rod 40. The third locking mechanism 32 and the axle 34 are disposed on the compressor 36 for rotatably connecting the compressor 36 to the angular selector 24. The fourth locking mechanism 38 buttresses the spring 68 on an end opposite the plug 60 such that the fourth locking mechanism 38 can selectively tune the force exerted by the spring 68 over a predetermined range. The spring 68 drives the rod 40 that is movably coupled to the plug 60 by a pin 70.

In a preferred embodiment, the rod 40 has the series of markers 50, such as colored bands, to indicate the force being exerted by the spring 68. As the rod 40 is moved towards the compressor 36, the spring 68 is compressed, corresponding to a greater spring force. Thus, a physician can use the markers 50 to visually determine how much force is being applied by the spring 68, and accordingly how much pressure the pressure pad 44 is exerting on the arterial wound. In a preferred embodiment, the spring 68 is calibrated to exert a range of pressure ranging from zero to 20 pounds per square inch (psi). More preferably, the markers 50 will designate specified pressures ranging from 5 to 15 psi.

The rod 40 engages the cam 42, which is used for adjusting the footprint of the pressure pad 44. The pressure pad 44 is coupled to the second rod 72 about an axle 52 protruding from a base portion 74. A pin 76 also protrudes from the base portion 74 to keep the pressure pad 44 properly aligned with the second rod 72 and the compressor 36. The cam 42 is used to rotate the pressure pad 44 about line A, thus permitting a full 360 degrees of access to the wound site by the pressure pad 44. As previously noted, the pressure pad 44 is preferably made of a transparent material for allowing the physician to precisely place the device 10 over the wound site. In another preferred embodiment, the pressure pad 44 is coated with a coagulating material (not shown) that will accelerate the hemostatic process on contact with the wound site.

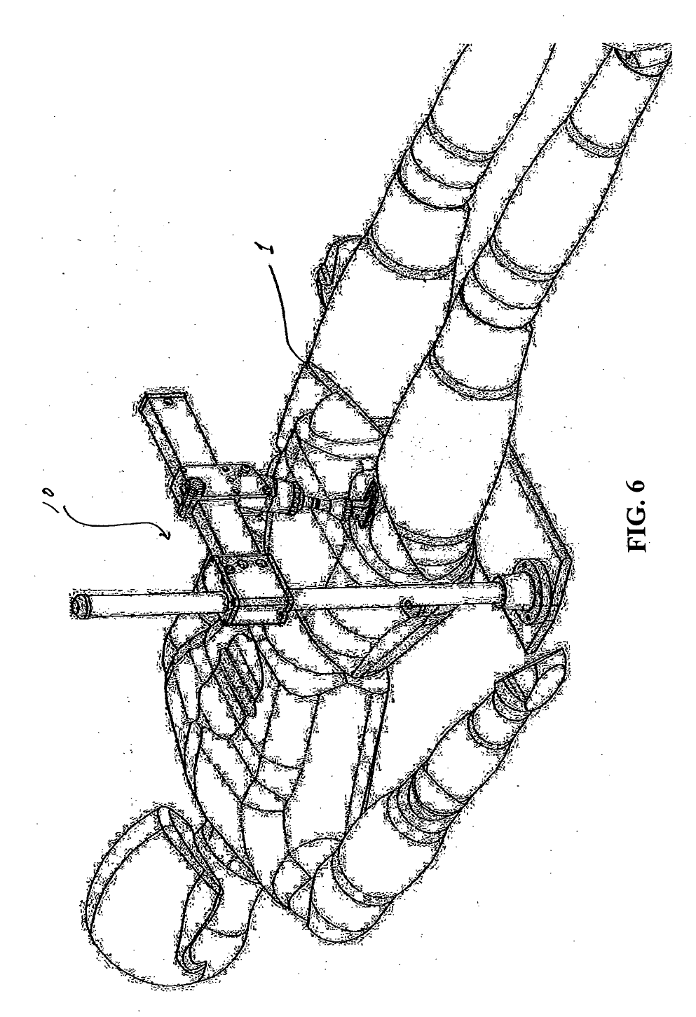

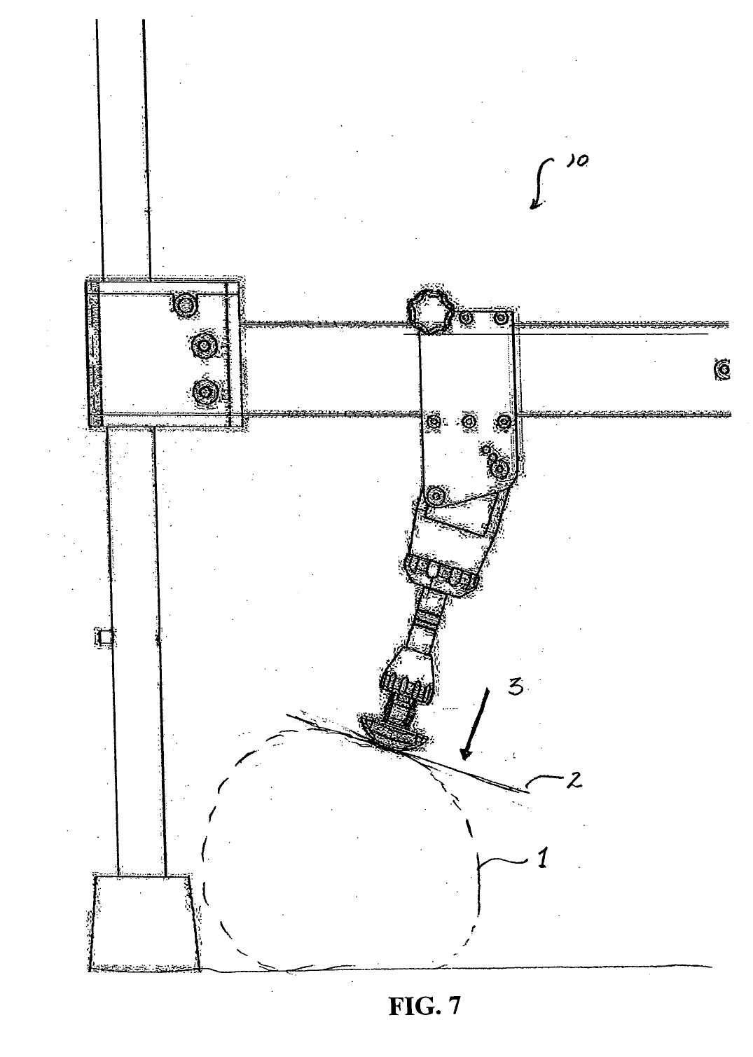

FIGS. 6 and 7 depict the device 10 of the present invention in use. FIG. 6 is a perspective view of the present invention in use with a typical patient 1 shown. FIG. 7 is a cross-sectional view of the present invention showing a cross-sectional portion of a patient's body 1 in phantom. As illustrated, the outline of the patient's body 1 is not planar in nature, but rather defines a number of planar surfaces such as a surface 2. The surface 2 in this example is tangential to the site of the arterial wound. Accordingly, the present invention 10 is adapted for directing a pressure along arrow 3 that is directly perpendicular to the surface 2, and thus optimized for inducing hemostasis in the artery.

In a preferred embodiment, the device 10 of the present invention is utilized according to the method described herein. In a first step, the arterial wound is disposed on the base 12 in the manner shown in FIGS. 6 and 7. The physician will then select the appropriate vertical height and horizontal height for the angular selector 30. The angle of the compressor 36 is then adjusted such that any spring force will be directed perpendicular to the wound site as shown in FIG. 7. The physician then manually stems the flow of blood from the wound while simultaneously adjusting the fourth locking mechanism 38 to apply a required spring force. The physician can then note the location of the markers 50 for an indication as to the amount of force/pressure that is being applied to the tops of his or her fingers. Once the position of the markers is noted, then the physician can remove his or her fingers, place the pressure pad 44 directly on the wound site, and readjust both the fourth locking mechanism 38 and the cam 42 such that the markers 50 are in the identical position as before. Once a hemostatic state is reached, the physician can decrease the pressure on the wound by compressing the spring 68 through the fourth locking mechanism 38, at which time the device 10 can be systematically removed from around the patient's body.

As described above, the present invention includes a device for inducing hemostasis in an artery, also referred to as a femoral clamp in a specific embodiment involving the femoral artery. Through novel design and function, the present invention permits attending medical personnel to mechanically curtail bleeding from an artery in a precise and controllable manner. In particular, by applying the pressure in a direction perpendicular to the patient's wound site, the present invention reduces the time needed for closing the artery. Moreover, as the device employs a number of movable members and locking mechanisms to attain many degrees of freedom, it is readily adaptable to a range of body types, including the most and least muscular among us.

It should be apparent to those skilled in the art that the above-described embodiments are merely illustrative of but a few of the many possible specific embodiments of the present invention. Numerous and various other arrangements can be readily devised by those skilled in the art without departing from the spirit and scope of the invention as defined in the following claims.

What is claimed is:

1. A device for inducing hemostasis in an artery, the device comprising:
a base;
a vertical member coupled to the base;
a horizontal member slidably coupled to the vertical member;
an angular selector slidably coupled to the horizontal member;
a compression arm rotatably coupled to the angular selector; and
a pressure pad coupled to the compression arm wherein the compression arm is selectively positioned along a horizontal axis, a vertical axis, and an angular position such that the pressure pad applies pressure to an artery.

2. The device of claim 1 wherein the angular selector provides an angular range between zero and sixty degrees through which the compression arm may be rotated.

3. The device of claim 1 wherein the compression arm comprises a compressor for generating a first pressure quantity.

4. The device of claim 1 wherein the compression arm comprises a cam for rotating the pressure pad.

5. The device of claim 3 wherein the compressor includes a spring buttressed on a first end by a plug, and kinetically engaged to a rod on a second end, the engagement between the spring and the rod set at a predetermined quantity corresponding to a spring force.

6. The device of claim 5 wherein the rod includes a plurality of markers for signifying a predetermined spring force.

7. The device of claim 4 wherein the cam is coupled to the pressure pad.

8. The device of claim 1 wherein the compression arm defines a longitudinal axis, and further wherein the pressure pad is rotatable about the longitudinal axis and pivotable with respect to the longitudinal axis.

9. The device of claim 1 further comprising a first locking mechanism integral to the horizontal member for locking the horizontal member at a selected position on the vertical member.

10. The device of claim 1 wherein the angular selector comprises a first portion that is slidably coupled to the horizontal member and a second portion that rotatably couples to the compression arm.

11. The device of claim 10 wherein the first portion includes a second locking mechanism for securing the position of the angular selector on the horizontal member.

12. The device of claim 10 wherein the second portion includes a third locking mechanism and an axle for securing the angular position of the compression arm relative to the horizontal member.

13. The device of claim 1 wherein the compression arm includes a fourth locking mechanism and a fifth locking mechanism for selecting an amount of force driven by the pressure pad.

14. The device of claim 1 wherein the pressure pad is comprised of a transparent material.

15. The device of claim 1 further comprising a coagulating material applied to the pressure pad.

16. The device of claim 1 wherein the pressure pad comprises a first region defining a substantially convex shape for engaging an arterial wall, and further wherein the pressure pad comprises a second region that is substantially planar for engaging an arterial wall.

17. A femoral artery clamp comprising:
a compression arm having a compressor for applying a first pressure and a cam for providing a second pressure;
a frame comprising a base, a vertical member coupled to the base, and a horizontal member slidably coupled to the vertical member; and
an angular selector defining a first portion slidably coupled to the horizontal member and further defining a second portion to which the compression arm is rotatably coupled.

18. The femoral artery claim of claim 17 further comprising a pressure pad coupled to the compression arm.

19. The femoral artery claim of claim 17 wherein the angular selector provides an angular range between zero and sixty degrees through which the compression arm may be rotated.

20. The femoral artery clamp of claim 17 further comprising a plurality of markers for signifying a predetermined spring force related to the first pressure.

21. The femoral artery clamp of claim 17 further comprising a first locking mechanism integral to the horizontal member for locking the horizontal member at a selected position on the vertical member.

22. The femoral artery clamp of claim 17 wherein the first portion includes a second locking mechanism for securing the position of the angular selector on the horizontal member.

23. The femoral artery clamp of claim 17 wherein the second portion includes a third locking mechanism and an axle for securing the angular position of the compression arm relative to the horizontal member.

24. The femoral artery clamp of claim 17 wherein the compression arm includes a fourth locking mechanism and a fifth locking mechanism for selecting an amount of force to be applied.

25. The femoral artery clamp of claim 18 wherein the pressure pad is comprised of a transparent material.

26. The femoral artery clamp of claim 18 further comprising a coagulating material applied to the pressure pad.

27. The femoral artery clamp of claim 18 wherein the pressure pad comprises a first region defining a substantially convex shape for engaging an arterial wall, and further wherein the pressure pad comprises a second region that is substantially planar for engaging an arterial wall.

28. A method of inducing hemostasis in an artery using a mechanical device, the method comprising:
providing a mechanical device;
applying manual pressure of a first quantity to a wound site using one or more fingers;
positioning the mechanical device over the one or more fingers such that a portion of the medical device is directed at the wound site;
adjusting the mechanical device to apply a pressure of a second quantity, the second quantity equal to the first quantity;
measuring the second quantity;
removing the one or more fingers from the wound site; and
guiding the mechanical device to the wound site.

29. The method of claim 28 further comprising the step of selecting an angle for the portion of the mechanical device such that it contacts the wound site normal to the surface of the wound site.

30. The method of claim 28 further comprising the step of applying a coagulating material to the second portion of the mechanical device to further induce hemostasis.

31. The method of claim 28 wherein the mechanical device includes a plurality of visual markers for measuring the second quantity.

32. The method of claim 28 further comprising the step providing a pressure pad to apply pressure.

33. The method of claim 28 wherein the step of adjusting the mechanical device to apply pressure of a second quantity includes rotating a cam mechanism to rotate a pressure pad in a selected manner to properly simulate manual pressure on the wound site.
